(19) United States
(12) Patent Application Publication
(10) Pub. No.: US 2019/0344121 A1
(43) Pub. Date: Nov. 14, 2019

(54) EXERCISE TRAINING ADAPTATION USING PHYSIOLOGICAL DATA

(71) Applicant: Mad Apparel, Inc., Redwood City, CA (US)

(72) Inventors: Barton S. Wells, Palo Alto, CA (US); Ankit Gordhandas, Sunnyvale, CA (US); Dhananja Pradhan Jayalath, San Francisco, CA (US)

(21) Appl. No.: 16/411,872

(22) Filed: May 14, 2019

Related U.S. Application Data
(60) Provisional application No. 62/671,309, filed on May 14, 2018.

(51) Int. Cl. A63B 24/00; A61B 5/024; A61B 5/0488; A61B 5/11; A61B 5/0402; A61B 5/00; A61B 5/22

(52) U.S. Cl. CPC A63B 24/0062; A61B 5/02438; A61B 5/0488; A61B 5/1118; A61B 5/0402; A61B 5/6804; A61B 2503/10; A61B 5/222; A63B 2024/0068; A63B 2024/0071; A63B 2230/04; A61B 2562/0219; A61B 5/486

(57) ABSTRACT

An exercise feedback system generates biofeedback based on physiological adaptations. The exercise feedback system processes physiological data from sensor-equipped garments worn by athletes while performing exercises. The exercise feedback system may use a trained model to determine classifications of segments of the physiological data. Classifications may represent a type of physiological adaptation, for example, power, strength, hypertrophy, endurance, or speed. Athletes can focus on one or more physiological adaptations, which may be based on a specific sport or training goal of an athlete. The exercise feedback system may also use other types of sensor data from the garments such as motion data or bioimpedance information. The exercise feedback system can generate biofeedback including metrics determined using the classifications. For example, the metrics indicate training load aggregated over multiple muscles or workouts, or the biofeedback may notify athletes regarding a risk of injury.

### CROSS-REFERENCE TO RELATED APPLICATIONS

This application claims the benefit of U.S. Provisional Application Ser. No. 62/671,309 filed 14 May 2018, which is incorporated in its entirety herein by this reference.

### BACKGROUND

This description generally relates to sensor-equipped athletic garments, and specifically to determining the type of exercise training adaptation using physiological data from the sensors.

Sensors record a variety of information about the human body. For example, electrocardiograph (ECG) electrodes can measure electrical signals from the skin of a person that are used to determine the person's heart rate. In addition, electromyography (EMG) electrodes can measure electrical activity generated by a person's muscles. EMG provides data associated with neuromuscular function of a person and ECG provides data associated with cardiovascular function. EMG signals associated with strength training exercises may differ from EMG signals associated with endurance training exercises. Thus, it is challenging for a system to generate useful metrics of athletic performance without distinguishing between different types of training. Since athletes playing different sports may have personalized training programs to improve performance based on the specific demands of the sport, it is desirable for systems to tailor metrics by taking into account the relevant types of training.

### SUMMARY

The goal of training and strength and conditioning in competitive athletics is to prepare an athlete for the demands of their sport and to improve performance. The trainable characteristics of an athlete (supported by American College of Sports Medicine) include, for example, power, strength, hypertrophy, anaerobic endurance, aerobic endurance and speed. Different sports require a different makeup of these trainable characteristics. For example, to improve performance in a 100 meter (100 m) sprint, athletes may focus their training on increasing power, strength and speed. However, to improve performance in the 10 kilometer (10 k) distance, athletes may focus their training on improving anaerobic and aerobic endurance. Using a combination of EMG and ECG sensors, and optionally motion data (e.g., accelerometer data) as an input, a method is described to classify adaptation types and/or determine the proportion of training adaptation types based on the type of neuromuscular and cardiovascular function.

The type of training (e.g., intensity, duration, frequency, and rest) determines the type of stress placed on the neuromuscular and cardiovascular systems. Over time as the body is repeatedly stressed with a specific type of training, the body will adapt to improve performance for that type of training. For example, in the case of the 100 m sprint athlete, repeated training at high intensity over short duration will adapt the athlete to increase power and strength. And in the case of the 10 k distance, training for longer durations at lower intensity will adapt the athlete to increase anaerobic and aerobic endurance.

Tailoring training based on the specific requirements for an individual athlete and their sport has proved to be difficult in conventional systems, largely due to the inability to understand what type of stresses are on each portion of the body during training. EMG sensors provide a measure of neuromuscular function and ECG sensors provide a measure of cardiovascular function. Using a combination of EMG sensors and ECG sensors the embodiments disclosed herein can categorize each athlete's training into the proportion of each adaptation type (e.g., power, strength, hypertrophy, endurance and speed) within a repetition, set, or session of exercise.

By leveraging a classification of the adaptation types, training can be further tailored for the athlete to match training to the specific types of stress required of the athlete for their sport. By classifying adaptation types metrics and relevant data may be provided to the athlete or coach. For example, the metrics can provide the amount of stress for different muscle groups for each adaptation type. The distribution of muscular stress for power and strength adaptation segments may be more important to the 100 m sprint athlete. Also, a model trained to predict injury risk for an athlete can leverage the training adaptation data to improve the predictability of the model. For example, features associated with power and strength adaptation types may be more predictive of injury risk for a 100 m sprint athlete, or certain features may have greater weight in the model for the power and strength adaptations. As another example, for a 10 k endurance athlete, specific features within the anaerobic endurance adaptation may have greater weight to improve injury predictability.

An exercise feedback system generates biofeedback based on physiological adaptations. The exercise feedback system processes physiological data from sensor-equipped garments worn by athletes while performing exercises. The physiological data may include EMG signals indicative of muscle activation levels. Additionally, the physiological data may include ECG signals indicative of heart rate. The exercise feedback system can use motion data from the sensors to identify segments of the physiological data representing periods of time during which the athletes were actively performing the exercises. In addition, the exercise feedback system can use bioimpedance data to determine noise levels or contact quality of the EMG and ECG sensors to the athlete's body. Bioimpedance data for athletic garments is further described in U.S. patent application Ser. No. 15/257,739.

The exercise feedback system may use a trained model to determine classifications of segments of the physiological data. Classifications may represent a type of physiological adaptation, for example, power, strength, hypertrophy, endurance, or speed. As used herein, physiological adaptations may include any changes to the athlete's physiology as result of exercise, e.g., growth in muscles or increase in cardiorespiratory capacity. In relation to training the model, embodiments of the system and method can create a training set upon applying a set of operations (e.g., signal processing operations, frequency domain operations, time domain operations, etc.) to a reference set of physiological data generated from one or more reference users and associated with the exercise(s) being analyzed. The model can be trained, using the training set, to determine, for each of a set of types of physiological adaptations (e.g., power, strength, hypertrophy, endurance, or speed), a probability that a given subset of physiological data is associated with one or more of the set of types of physiological adaptations.

Athletes can focus on training for one or more physiological adaptations, which may be based on a specific sport or training goal of an athlete. For example, long distance runners focus on endurance training, while Olympic lifters focus on power and strength training. The exercise feedback system can generate biofeedback including metrics determined using the classifications. For example, the metrics indicate training load aggregated over multiple muscles or workouts, or the biofeedback may notify athletes regarding a risk of injury.

### BRIEF DESCRIPTION OF DRAWINGS

FIG. 1 is a diagram of a system environment for providing feedback based on physiological adaptations, according to an embodiment.

FIG. 2 is a diagram of a sensor-equipped athletic garment, according to an embodiment.

FIG. 3 is a block diagram of an exercise feedback system, according to an embodiment.

FIG. 4 is a diagram of example physiological data, according to an embodiment.

FIG. 5A is a data flow diagram of an adaptation model, according to an embodiment.

FIG. 5B is another data flow diagram of an adaptation model, according to an embodiment.

FIG. 5C illustrates a decision tree for use in determining parameters by an adaptation model, according to an embodiment.

FIG. 6 is a diagram illustrating differences between types of physiological adaptations, according to an embodiment.

FIG. 7 is a flowchart of a process for providing biofeedback, according to an embodiment.

FIG. 8 is a flowchart of another process for providing biofeedback, according to an embodiment.

The figures depict embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### I. System Overview

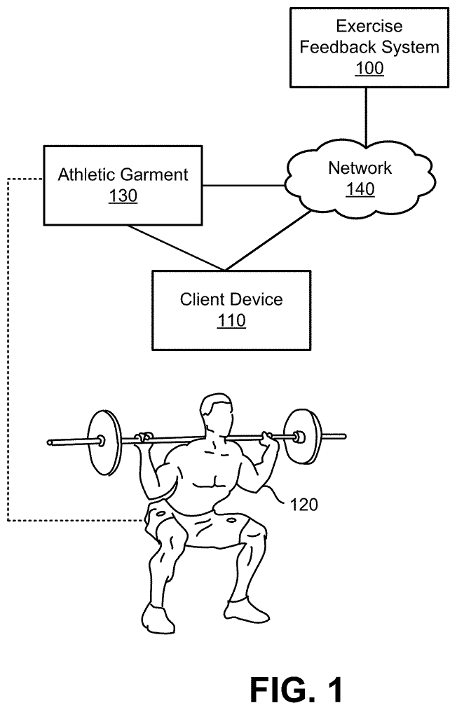

FIG. 1 is a diagram of a system environment for providing feedback based on physiological adaptations, according to an embodiment. The system environment includes an exercise feedback system 100, client device 110, and athletic garment 130 communicatively coupled together via a network 140. Users 120 of the exercise feedback system 100 may also be referred to herein as “athletes” or “coaches.” In other embodiments, different and/or additional entities can be included in the system architecture.

The client device 110 is a computing device capable of receiving user input as well as transmitting and/or receiving or collecting data via the network 140. A client device 110 is a device having computer functionality, such as a smartphone, personal digital assistant (PDA), a mobile telephone, tablet, laptop computer, desktop computer, a wearable computer (such as a smart watch, wrist band, arm band, chest band, or the like), or another suitable device. In one embodiment, a client device 110 executes an application allowing a user of the client device to interact with the exercise feedback system 100. For example, a client device 110 executes a browser application to enable interaction between the client device 110 and the exercise feedback system 100 via the network 140. In another embodiment, a client device 110 interacts with the exercise feedback system 100 through an application programming interface (API) running on a native operating system of the client device, such as IOS® or ANDROID™.

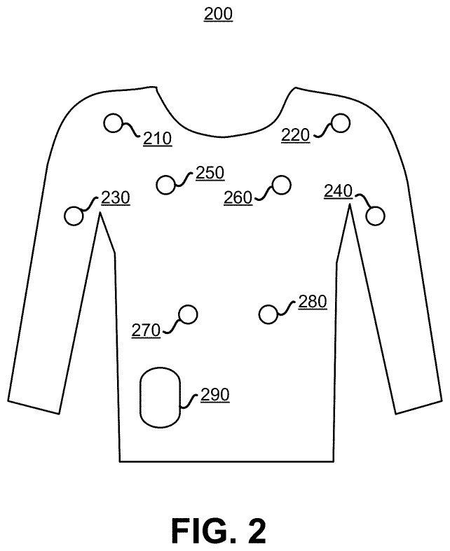

An athlete 120 wears the athletic garment 130 (further described below with reference to FIG. 2) while performing exercises. The athletic garment 130 records physiological data, e.g., EMG signals or heart rate data, of the athlete. Based on the physiological data, the exercise feedback system 100 generates biofeedback customized for the athlete. The biofeedback may be determined using physiological data (e.g., muscle stress measurements) corresponding to any muscle, portion of a muscle, or set of muscles of the athlete. For example, the athlete's biceps may include a left and right biceps muscle, and the biceps may be part of a group of upper body muscles. As described herein, a muscle includes soft tissue for the purpose of producing force or motion of the human body. Further, a coach of the athlete can view the biofeedback on a client device 110 and provide additional feedback for the athlete. In some embodiments, the exercise feedback system 100 can communicate feedback using audio output or haptic feedback of the client device 110 or athletic garment 130.

In various embodiments, the exercise feedback system 100 generates biofeedback based on physiological adaptations, which may provide context to support personalizing training for athletes to reduce injury risk or focus on particular areas of performance. For instance, the exercise feedback system 100 may evaluate injury risk by monitoring stresses experienced by particular muscles over a period of time. For example, physiological adaptations can be used to weight the stresses of particular muscle groups in an injury risk model based on the type of adaptation. One set of features (e.g., muscle stress, activation, sequence, balance, etc.) may show higher correlation to injury risk for an athlete with a higher proportion of power and strength adaptation training. A different set of features may show higher correlation to injury risk for an athlete with a higher proportion of endurance adaptation training. Additionally, using physiological adaptations, the exercise feedback system 100 may determine biofeedback used to improve training for a specific type of sport or goal of an athlete. As used herein, types of physiological adaptations may include, e.g., power, strength, hypertrophy, speed, and endurance. In other embodiments, a different number or other types of physiological adaptations may be used. An athlete training for long distance running (e.g., a marathon) may want to focus on training for endurance, while a different athlete training for weight-lifting may want to focus on training for strength or hypertrophy.

### II. Example Athletic Garment

FIG. 2 is a diagram of a sensor-equipped athletic garment 200, according to an embodiment. The athletic garment 200 includes sensors that contact the skin of an athlete wearing the athletic garment 200. For example, the sensors can be electrodes that measure electromyography (EMG) signals (electrical signals caused by muscle cells), electrocardiograph (ECG) signals (electrical signals caused by depolarization of the user's heart muscle in particular) also referred to as heart rate data, electrical signals modified by the interface between the electrodes and tissue of the user (also referred to as bioimpedance data), electroencephalograph (EEG) signals, magnetoencephalograph (MEG) signals, among other types of signals associated with physiological data. The sensors may also include other types of sensors such as accelerometers and gyroscopes (which generate motion data based on the athlete's movement), temperature sensors, pressure sensors, humidity sensors, geographical location sensors, etc. The sensors generate physiological data of the athlete based on the measured signals. The sensors are communicatively coupled to a processing unit 290. The processing unit 290 can aggregate and analyze the physiological data from the sensors. The processing unit 290 can also provide the physiological data to the client device 110 or exercise feedback system 100 via the network 140.

In the embodiment shown in FIG. 2, the athletic garment 200 includes eight sensors that record physiological data from the athlete's muscles nearby each sensor. In particular, sensors 210 and 220 located on the right and left shoulder of the athletic garment 200 can record EMG signals associated with the athlete's deltoid muscles. Sensors 230 and 240 located on the right and left sleeves of the athletic garment 200 can record EMG signals associated with the athlete's triceps and/or bicep muscles. Sensors 250 and 260 located on the right and left chest of the athletic garment 200 can record EMG signals associated with the athlete's pectoral muscles. Sensors 270 and 280 located on the right and left abdomen of the athletic garment 200 can record EMG signals associated with the athlete's abdominal and oblique muscles. Though the athletic garment 200 shown in FIG. 2 includes eight sensors and the processing unit 290, in other embodiments, the athletic garment 200 can include any number of sensors or other types of components or electronics at any location or configuration within the athletic garment 200.

It should be noted that while the athletic garment 200 shown in FIG. 2 is a long sleeve shirt, the principles described herein apply equally to any garment, including but not limited to a short sleeved shirt, a tank top, pants, shorts (e.g., the athletic garment 130 shown in FIG. 1), or any other suitable garment. In embodiments where the athletic garment is a pant or shorts, sensors of the athletic garment can record EMG signals associated with from muscles on an athlete's lower body, e.g., inner and outer quadriceps (also referred to as “quads”), gluteus maximus (also referred to as “glutes”), hamstrings, calves, and the like.

### III. Example Exercise Feedback System

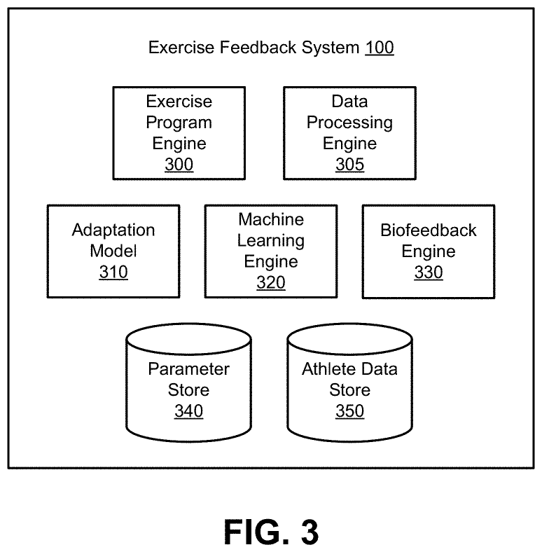

FIG. 3 is a block diagram of the exercise feedback system 100, according to an embodiment. The exercise feedback system 100 includes an exercise program engine 300, data processing engine 305, adaptation model 310, machine learning engine 320, biofeedback engine 330, parameter store 340, and athlete data store 350. In other embodiments, the exercise feedback system 100 may include additional, fewer, or different components for various applications, which are not shown for purposes of clarity.

The exercise program engine 300 manages exercise programs for athletes of the exercise feedback system 100. An exercise program may include one or more workouts or exercise sets (also referred to as a “set”), where a set includes a number of repetitions of an exercise to be performed in sequence. In some embodiments, an athlete registers on the exercise feedback system 100 by completing an onboarding process during which the exercise program engine 300 receives user information or physiological data associated with the athlete. The exercise program engine 300 may store the received user information or physiological data in the athlete data store 350. User information includes, for example, demographic data (e.g., age, gender, ethnicity, etc.), geographic data, exercise related data (e.g., sports played, a specific position for sport, or sports team information), or physical attributes (e.g., height or weight). Physiological data may include data generated by the sensors of an athletic garment, e.g., for onboarding or calibration.

The data processing engine 305 processes physiological data generated by sensors of an athletic garment (e.g., athletic garment 130 or 200 shown in FIGS. 1-2) for determining biofeedback. The data processing engine 305 can receive the physiological data from a client device 110 or the processing unit 290 of the athletic garment 200. In some embodiments, the processing unit 290 of the athletic garment 200 pre-processes the physiological data by performing noise filtering techniques. The data processing engine 305 may additionally process the physiological data by performing noise filtering techniques. The data processing engine 305 may also normalize physiological data based on calibration data for a given user, type of exercise, type of physiological adaptation, or other types of reference data.

The data processing engine 305 may use EMG signals of physiological data to determine metrics including, for example, muscle activation data, training load, or muscle stress. The EMG signals may vary based on parameters associated with the athlete wearing the athletic garment, e.g., differences in the physiology between multiple types of muscles influences the physiological data representing contraction of the muscles. For instance, the glutes and quads differ in the number of muscle fibers, fiber size, fiber type distribution (e.g., slow twitch or fast twitch), and thickness of adipose tissue between the muscle tissue and skin surface. The amplitude of EMG signals (e.g., muscle stress) may be proportional to the muscle fiber size and/or inversely proportional to the amount of fat between the muscle and the portion of skin.

The data processing engine 305 may determine a quality of physical contact between a sensor of an athletic garment and an athlete's skin based on bioimpedance data, which is generated by another sensor or the same sensor. Thicker hair on the athlete's skin or skin dryness may result in poor contact quality, and thus reduce the amplitude of (or otherwise modify) EMG signals generated by a sensor, or introduce additional noise in the EMG signals. In some embodiments, the amplitude of physiological data generated by a sensor and the signal to noise ratio is inversely proportional to the bioimpedance between the sensor and the skin of the athlete. Responsive to determining that the quality of physical contact during a period of time is less than a threshold quality (e.g., indicative of poor contact quality), the data processing engine 305 may exclude or modify the physiological data received during the period of time from generation of metrics or biofeedback.

In some embodiments, the data processing engine 305 uses at least one of physiological data and motion data received from motion sensors of an athletic garment to determine whether an athlete wearing the athletic garment is actively training or performing a particular type of exercise. In addition to resting between sets, the athlete may also intermittently rest between repetitions or otherwise temporarily interrupt or pause an exercise, e.g., to adjust the athletic garment. By classifying active segments (e.g., periods of time) and inactive segments in physiological data, the data processing engine 305 may account for discrepancies in the data, for example, filtering out noise from inactive periods during which the athlete is not performing an exercise. In some embodiments, the data processing engine 305 disregards physiological data, for determining metrics of a specific physiological adaptation or muscle, generated during a given exercise responsive to determining that the given exercise is not directed to training that specific physiological adaptation or muscle. For instance, physiological data of the glutes and quads generated while the athlete is performing certain upper body exercises may not be normalized or accumulated for determining metrics of the lower body muscles.

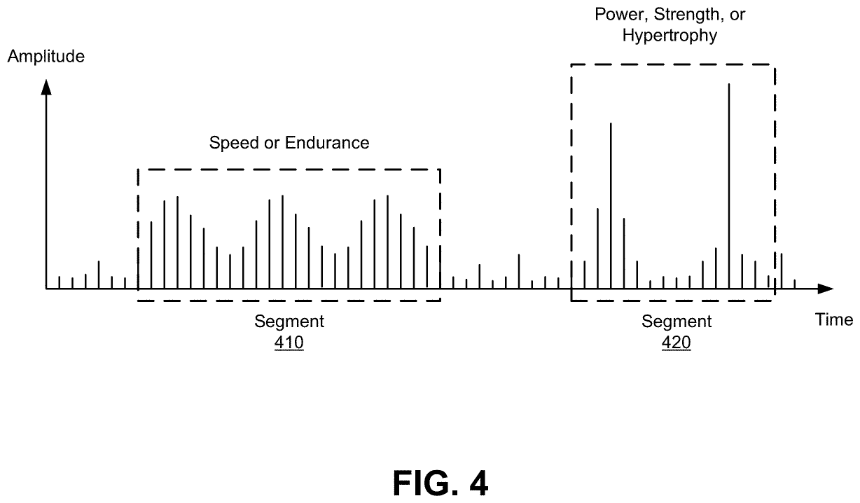

The adaptation model 310 classifies physiological data based on types of physiological adaptation. In particular, the adaptation model 310 may determine at least one classification of physiological adaptation for one or more segments of physiological data. The segments may be contiguous portions or subsets of the physiological data representing EMG signals generated by the sensors during different periods of time, which is further described with reference to FIG. 4.

In some embodiments, the adaptation model 310 determines adaptation parameters of an input physiological data and uses the adaptation parameters to evaluate the physiological data. The adaptation parameters may describe features such as frequency or amplitude of the physiological data, for example, one or more of EMG signals, ECG signals, and motion data (e.g., acceleration or gyroscope data). The adaptation model 310 may use the adaptation parameters to determine a probability that a given segment (e.g., subset of the physiological data) is associated with a specific one (or more) of the types of physiological adaptation. The adaptation model 310 stores adaptation parameters in the parameter store 340 and may also retrieve previously determined adaptation parameters from the parameter store 340, e.g., to use as feedback.

In some embodiments, the adaptation model 310 aggregates segments according to their respective type of physiological adaptation for determining metrics for athletes. Thus, the exercise feedback system 100 may accumulate training loads (EMG activation over time) for the aggregated segments to determine aggregate training loads for specific muscles and specific physiological adaptations. Responsive to determining that a ratio of aggregate training load of one muscle group over another is greater than a threshold for a certain period of time, and for a specific adaptation type, the exercise feedback system 100 may determine that the athlete has a greater risk of injury.

In some embodiments, the machine learning engine 320 uses one or more machine learning techniques to train an adaptation model 310. The machine learning engine 320 may use reference physiological data as training data for training the adaptation model 310. For instance, the reference physiological data is generated by sensors (e.g., of an athletic garment) on a population of users who performed exercises, and processed with one or more operations (e.g., signal processing operations, windowing, filtering, operations in the frequency domain, operations in the time domain, etc.) to create a training dataset. The training data may be labeled, for example, associating a certain training data set with one or more specific types of physiological adaptation (e.g., power, strength, hypertrophy, endurance, or speed), types of exercise, etc. The training data may also include reference motion data, e.g., a motion profile indicating proper (or improper) form of a certain exercise. During training, the adaptation model 310 may determine features of the training data such as reference parameters, which may be stored in the parameter store 340. A reference parameter may describe one or more attributes of sensor data, e.g., an expected peak amplitude or frequency of a data signal, a particular pattern in the data signal, or certain thresholds of amplitude or width of peaks in the data signal. Example reference parameters are described below with reference to FIG. 4. The trained model can thus output a probability that a given subset of physiological data is associated with one or more of the set of types of physiological adaptations, types of exercises, etc.

The biofeedback engine 330 generates or updates user interfaces to present biofeedback via client devices 110 to athletes, coaches, or other persons. Biofeedback may indicate a metric of athletic performance such as a percentage value, a Boolean value (e.g., satisfactory or unsatisfactory), or any suitable type of value. Metrics may be aggregated for multiple muscles, over multiple workouts, based on adaptation type or in any other suitable manner. In some embodiments, the biofeedback engine 330 generates biofeedback based on context, for example, a specific type of physiological adaptation, exercise, muscle or muscle group, repetition, set, or load (e.g., based on a product of a number of repetitions and corresponding weight lifted by an athlete).

In some embodiments, the biofeedback engine 330 generates a graphical depiction of muscles of an athlete for presenting biofeedback. In particular, the biceps and quads (among other types of muscles) may be overlaid on the arm and leg portions, respectively, of a human body graphic (e.g., resembling a silhouette, avatar, or the like) of the athlete. The biofeedback engine 330 may present metrics of muscles by dynamically updating a color or size of the graphic depiction of the corresponding muscle. For instance, as the muscle stress measurement of a given muscle increases, a graphic of the given muscle becomes a brighter color or increases in size to illustrate that the given muscle is being contracted for an exercise. Thus, the athlete can view a real-time progression of one or more muscles that increase or decrease in activation levels, stress, or training load throughout stages of an exercise.

In some embodiments, the biofeedback engine 330 communicates a risk of injury, e.g., via a push notification presented by a client device 110. The biofeedback engine 330 may notify an athlete of the risk of injury after an exercise or while an athlete is exercising, so that the athlete can adjust exercise training to avoid the injury or reduce the risk. The biofeedback engine 330 may provide context with a notification of injury risk, for example, indicating a type of risk (e.g., over stressing a specific muscle or joint), severity of the risk, or remedial action such as performing stretches, icing or heating a muscle, resting, or switching to a different type of physiological adaptation for training.

In some example use cases, an athlete using the exercise feedback system 100 is part of an athletic team including multiple athletes, coaches, or other personnel. The biofeedback engine 330 may generate biofeedback including a comparison between the athlete and another athlete of the same team. The biofeedback may present a comparison of athletes' performance categorized based on physiological adaptation. Furthermore, the biofeedback engine 330 may provide biofeedback for presentation to a coach of the team. The biofeedback engine 330 may send biofeedback based on aggregate metrics of the team to a client device 110 of the coach, or may flag individual team members based on different metrics and may identify an athlete to be at risk of injury, beneficially allowing the coach to intervene before injury occurs.

In some embodiments, some or all of the functionality of the exercise feedback system 100 may be performed by or implemented within a client device 110 or a processing unit 290 (of the athletic garment 200 shown in FIG. 2). For example, the client device 110 or processing unit 290 stores one or more pre-determined adaptation models 310 to process physiological data. This can be advantageous because the client device 110 or processing unit 290 may not always have a network connection while an athlete is exercising (e.g., the athlete's gym does not have internet available). Thus, the exercise feedback system 100 can determine and analyze biofeedback locally on the client device 110 or processing unit 290 without having to upload (e.g., in real time) the physiological data to a server of the exercise feedback system 100. The client device 110 or processing unit 290 may upload processed data or generated metrics to the exercise feedback system 100 at a later time, for instance, after an exercise.

### IV. Example Adaptation Model

FIG. 4 is a diagram of example physiological data, according to an embodiment. The example physiological data includes an EMG signal generated by sensors of an athletic garment worn by an athlete. The diagram plots the amplitude of the EMG signal over time. As shown by the dotted lines, the exercise feedback system 100 may determine a first segment 410 and a second segment 420 of the EMG signal. Additionally, the exercise feedback system 100 may determine a first and second classification of the first and second segment, respectively, by using the adaptation model 310 to predict one or more physiological adaptations associated with the segments.

In some embodiments, the adaptation model 310 classifies segments as one or more physiological adaptations by identifying one or more features from input data. The features, or reference parameters learned from training, may include a certain pattern (e.g., repeating for at least a particular duration of time or number of instances), or attributes detected in motion data or EMG data. In the example shown in FIG. 4, the exercise feedback system 100 may identify the first segment 410 and the second segment 420 as active segments during which an athlete is exercising. Additionally, the exercise feedback system 100 may determine that the intermediate portion of data between the first segment 410 and the second segment 420 is not an active segment because the average amplitude of the intermediate portion is less than a threshold. The intermediate portion may correspond to a period of time when the athlete was resting between exercises, and thus the exercise feedback system 100 does not necessarily need to classify data from that period of time.

The adaptation model 310 may determine that the EMG data during the time period of the first segment 410 represents a periodic signal having a given period and amplitude (e.g., peak amplitude or averaged over the duration of the segment). Further, the adaptation model 310 may compare the given period or amplitude to a reference period or amplitude, respectively, of a reference data generated by sensors monitoring a user performing exercise for one or more specific types of physiological adaptation. Responsive to determining that the given period is within a threshold error from the reference period, and/or determining that the given amplitude is within a threshold error from the reference amplitude, the adaptation model 310 may determine that the first segment 410 likely includes EMG data representing training for the one or more specific types of physiological adaptation.

Following in the above example, the reference data may be associated with an exercise having a speed or endurance type of physiological adaptation. For instance, the exercise is a treadmill run and the reference data includes EMG signal of lower body muscles of an athlete running on a treadmill. Accordingly, the adaptation model 310 determines that the first segment 410 should be classified with the speed or endurance type of physiological adaptation.

In some embodiments, the adaptation model 310 determines predictions of physiological adaptation by determining a rate of change of physiological data. Typically, power, strength, or hypertrophy types of exercises involve short bursts of muscle exertion, e.g., to lift a certain amount of weight. Thus, EMG signals generated by sensors tracking athletes performing these types of exercises may exhibit greater rates of change relative to EMG signals for speed or endurance types of exercises. In the example shown in FIG. 4, responsive to determining that a rate of change of the EMG signal in the second segment 420 is greater than a threshold rate, the adaptation model 310 determines that the second segment 420 should be classified with the power, strength, or hypertrophy type of physiological adaptation.

In some embodiments, the adaptation model 310 classifies a segment as a power type of physiological adaptation responsive to one or more of (i) determining that the EMG signal is greater than 100% of a reference EMG parameter (e.g., reference parameter set by the calibration process, where the reference parameter may indicate an expected peak value), (ii) determining that peaks in the EMG signal have less than a threshold width, (iii) determining that the rate of change of the EMG signal during peaks is greater than a threshold rate, (iv) determining that an accumulation of EMG signal over the duration of the peak is greater than a threshold value, (v) determining that a corresponding motion sensor data signal indicates at least a threshold acceleration (e.g., occurring simultaneously during at least a portion of the peaks in EMG signal), and (vi) determining that a heart rate signal has at least a threshold derivative and/or peak changes. The exercise feedback system 100 may determine (or update) the reference EMG parameter during calibration, user profile information, and/or reference data.

In some embodiments, the adaptation model 310 classifies a segment as a strength type of physiological adaptation responsive to one or more of (i) determining that the EMG signal is greater than a first threshold and less than the reference parameter (e.g., approximately 75% of the reference parameter), (ii) determining that peaks in the EMG signal have greater than a threshold width, (iii) determining that the rate of change of the EMG signal during peaks is less than a threshold rate, (iv) determining that the active duration or number of detected reps is less than a threshold duration or number, (v) determining that a corresponding motion sensor data signal indicates less than a threshold acceleration (e.g., occurring simultaneously during at least a portion of the peaks in EMG signal), and (vi) determining that a heart rate signal has at least a threshold derivative and/or peak changes in heart rate.

In some embodiments, the adaptation model 310 classifies a segment as a hypertrophy type of physiological adaptation responsive to one or more of (i) determining that the EMG signal is greater than a second threshold lower than the first threshold with respect to the strength type of physiological adaptation (e.g., approximately 65% of the reference parameter) and (ii) determining that the active duration or number of detected reps is greater than a threshold duration or number.

In some embodiments, the adaptation model 310 classifies a segment as an endurance type of physiological adaptation responsive to one or more of (i) determining that the EMG signal is less than the second threshold, (ii) determining that the EMG signal peak levels are consistent and repeating (e.g., responsive to determining that the Wiener entropy of a spectrum of the physiological data is less than a threshold value), and (iii) determining that variation in heart rate is less than a threshold rate or indicates slow gradual changes above a threshold value (e.g., above 40% of athlete's max heart rate). EMG signals having peaks less than a third threshold (e.g., approximately 35% of the expected peak value) may be indicative of aerobic endurance training, while EMG signals having peaks between the second and third threshold (e.g., inclusive) may be indicative of anaerobic endurance training.

In some embodiments, the adaptation model 310 classifies a segment as a speed type of physiological adaptation responsive to one or more of (i) determining that the EMG signal peaks repeatedly at least at a threshold frequency (e.g., determining that the Wiener entropy of the spectrum is less than a threshold value) and (ii) determining that motion data indicates repeated patterns in acceleration data at consistent frequency and/or having less than a threshold duration in time.

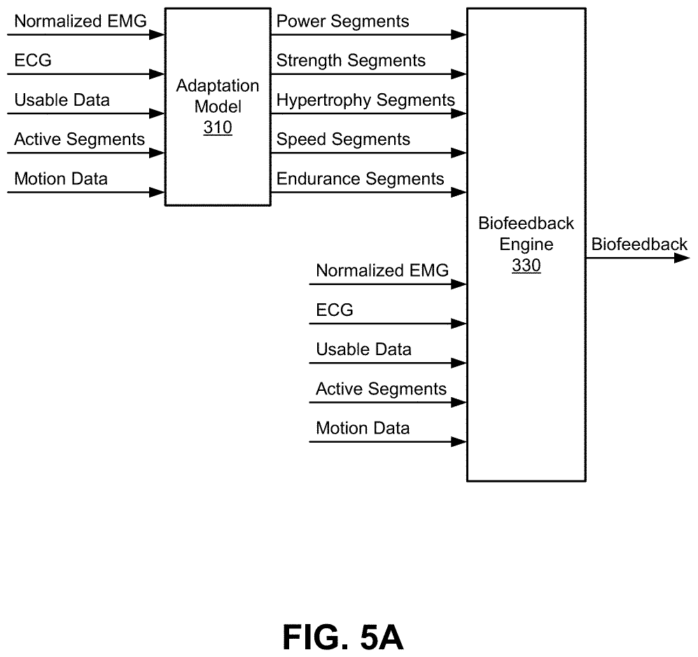

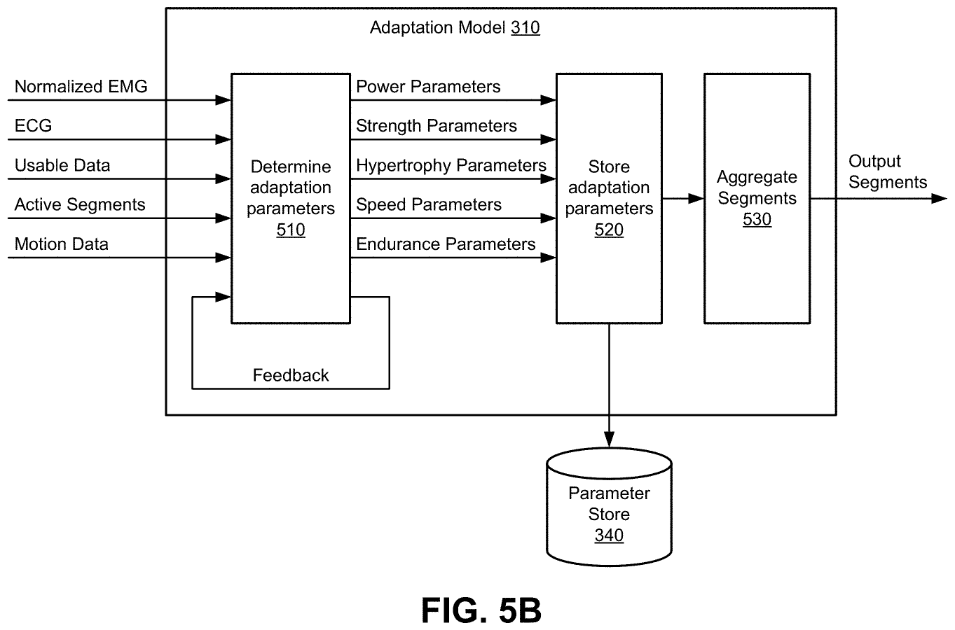

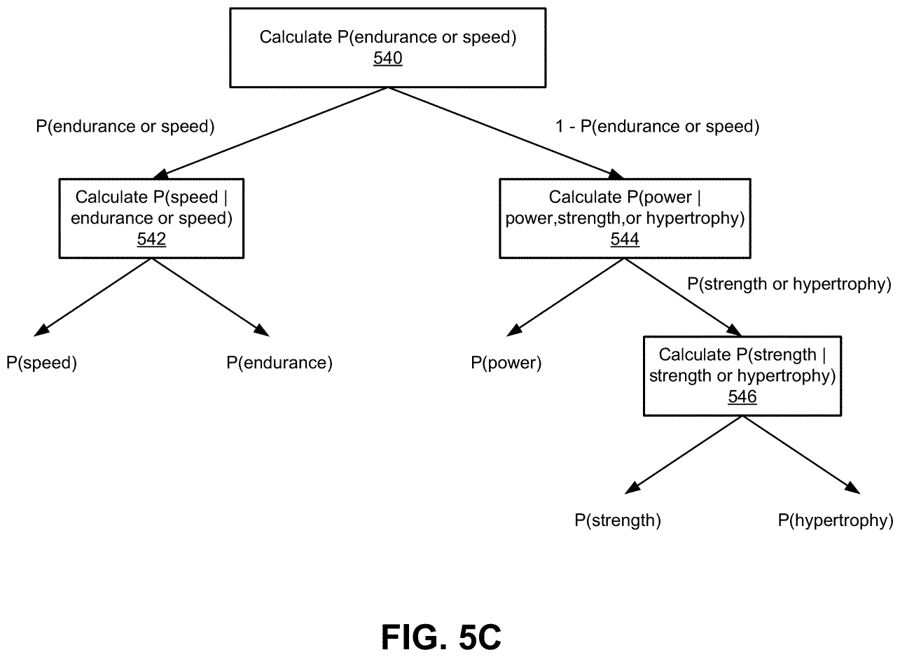

FIG. 5A is a data flow diagram of an adaptation model 310, according to an embodiment. In the illustrated example, the adaptation model 310 receives inputs including one or more of EMG signals, ECG signals, usable data, active segments, and motion data, among other types of input such as heart rate, video data, or body temperature data. The EMG or ECG signals may be normalized using calibration data of a given athlete. As previously described, the data processing engine 305 may pre-process physiological data generated by sensors of an athletic garment. In particular, the data processing engine 305 can determine usable data by extracting segments of the physiological data having at least a threshold quality of physical contact with skin of the athlete. Furthermore, the data processing engine 305 can determine active segments using the motion data and/or EMG data.

The adaptation model 310 uses the inputs to classify the active segments and usable data as being associated with one or more physiological adaptations. In some embodiments, the adaptation model 310 outputs any number of classified power segments, strength segments, hypertrophy segments, speed segments, and endurance segments. The biofeedback engine 330 may receive the output from the adaptation model 310 and generate biofeedback based on the classified segments. Additionally, the biofeedback engine 330 may generate biofeedback further using any number of the inputs to the adaptation model 310. For example, the biofeedback determines a measure of stress for each muscle group for the session by aggregating the stress corresponding to the segments of each adaptation type. In another embodiment, the biofeedback includes metrics for segments of a certain classification accompanied with context such as the exercise form of the athlete (e.g., based on EMG and motion data) or the athlete's heart rate while training.

FIG. 5B is another data flow diagram of the adaptation model 310, according to an embodiment. In some embodiments, to classify segments of physiological data, the adaptation model 310 determines 510 adaptations parameters for one or more types of the physiological adaptations. The adaptation model 310 may store 520 the adaptation parameters in the parameter store 340. In addition, the adaptation model 310 can use previously determined adaptation parameters (e.g., retrieved from the parameter store 340) as feedback for determining subsequent parameters. To identify segments, the adaptation model 310 may divide physiological data into epochs (e.g., subsets of data) each representing data for a given period of time. The adaptation model 310 can use a state machine to determine a start epoch and an end epoch for a particular type of physiological adaptation. For instance, among a data set spanning ten epochs, the adaptation model 310 determines that the second through fifth epoch represent data generated when an athlete was training for endurance. Epochs including noisy data or outliers may be filtered out by the data processing engine 305.

The adaptation model 310 aggregates segments 530 of physiological data according to their respective classifications. For example, the aggregates segments for at least one of power, strength, hypertrophy, speed, and endurance. The aggregated segments are output, for example, to the biofeedback engine 330 for generating biofeedback, which may include metrics of an athlete's aggregate training load or contribution of muscles categorized by type of physiological adaptation. The metrics may allow an athlete to determine whether the athlete is focusing on a desired type of training, to identify muscles that are being overexerted, or to identify muscles that are being under-utilized and need further training.

FIG. 5C illustrates a decision tree for use in determining parameters by the adaptation model 310, according to an embodiment. In the illustrated example, the adaptation model 310 classifies segments (or epochs) of physiological data by determining probabilities that the segments are associated with speed, endurance, power, strength, or hypertrophy training. In examples, probabilities for segment classification are determined based on a set of features, which can include or be derived from one or more of: EMG frequency spectrum, rate of change of total EMG, rate of change of EMG of a specific muscle, rate of change of accelerometer signal parameter, duration of activity, repetitions associated with activity (e.g., number and amplitude of EMG and/or accelerometer-derived repetitions), maximum EMG signal parameter (e.g., amplitude) during a period of time, maximum accelerometer signal parameter (e.g., amplitude) during a period of time, average EMG signal parameter (e.g., amplitude) during a period of time, average accelerometer signal parameter (e.g., amplitude) during a period of time, pattern of repetition in EMG and/or accelerometer-derived signals (e.g., from autocorrelation calculations, from spectrum-based determinations, etc.), duration of time associated with different intensity levels, and other parameters.

In the decision tree, the adaptation model 310 calculates 540 P(endurance or speed), which represents the probability that a given segment should be classified as endurance or speed. GivenP(endurance or speed), the adaptation model 310 calculates 542 P(speed|endurance or speed), the probability that the given segment should be classified as speed. Thus, the adaptation model 310 calculates the probability that the given segment should be classified as speed as P(speed)=P(endurance or speed)*P(speed|endurance or speed) The adaptation model 310 calculates the probability that the given segment should be classified as endurance as P(endurance)=P(endurance or speed)*(1−P(speed|endurance or speed)).

On the other branch of the example decision tree, the probability that the given segment should be classified as power, strength, or hypertrophy is P(power, strength, or hypertrophy)=1−P(endurance or speed) Given P(power, strength, or hypertrophy), the adaptation model 310 calculates 544 P(power|power, strength, or hypertrophy), the probability that the given segment should be classified as power. Thus, the probability that the given segment should be classified as strength or hypertrophy P(strength, or hypertrophy)=P(power, strength, or hypertrophy)*(1−P(power|power, strength, or hypertrophy)) Given P(strength or hypertrophy), the adaptation model 310 calculates 546 P(strength|strength or hypertrophy), the probability that the given segment should be classified as strength. Thus, the probability that the given segment should be classified as hypertrophy is P(hypertrophy)=P(strength or hypertrophy)*(1-P(strength|strength or hypertrophy)

The adaptation model 310 may store the parameters for each segment or epoch in a table, array, or other suitable data structure in the parameter store 340.

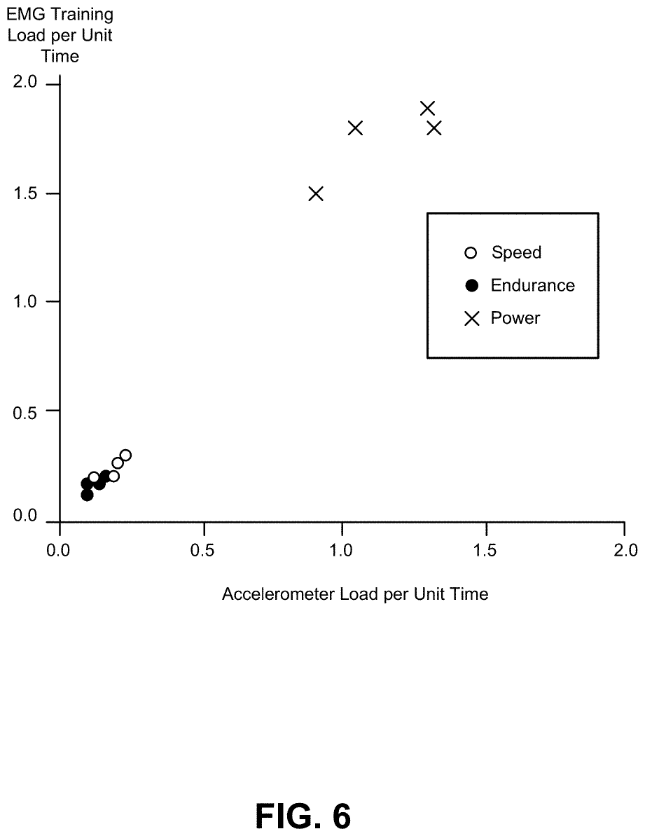

FIG. 6 is a diagram illustrating differences between types of physiological adaptations, according to an embodiment. The example diagram include metrics for segments of speed, endurance, and power types of physiological adaptations. Particularly, the x-axis indicates accelerometer load per unit time (e.g., second or minute), and the y-axis indicates the sum of EMG activation per unit time or training load per unit time. As shown by the plotted data points, physiological data classified as power have greater EMG training load per unit time and accelerometer load per unit time, in comparison to physiological data classified as speed or endurance. The adaptation model 310 can be trained to learn and identify these different features between types of physiological adaptations.

### V. Example Process Flows

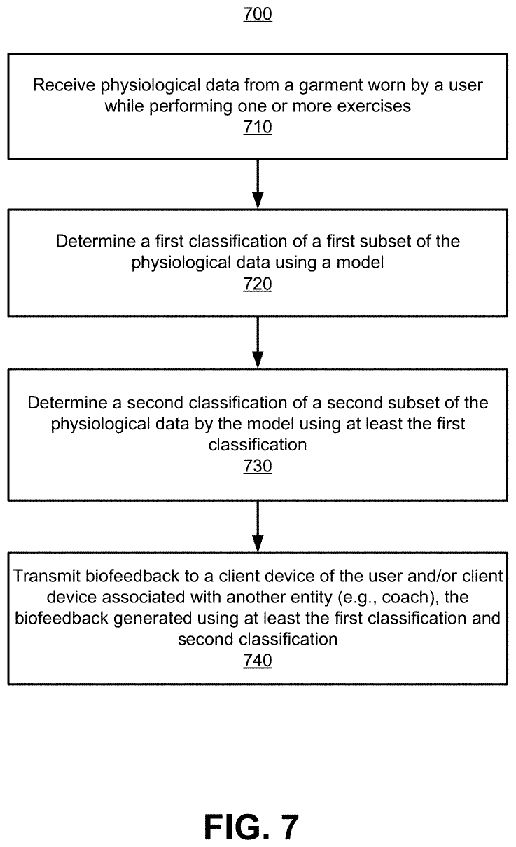

FIG. 7 is a flowchart of a process 700 for providing biofeedback, according to an embodiment. In some embodiments, the process 700 is performed by the exercise feedback system 100—e.g., modules of the exercise feedback system 100 described with reference to FIG. 3—within the system environment in FIG. 1. The process 700 may include different or additional steps than those described in conjunction with FIG. 7 in some embodiments or perform steps in different orders than the order described in conjunction with FIG. 7.

In one embodiment, the exercise feedback system 100 receives 710 physiological data from a garment (e.g., garment 200 shown in FIG. 2) worn by a user. The physiological data describes muscle activation of a set of muscles of the user while performing one or more exercises. Additionally, the garment includes a set of sensors configured to generate the physiological data. The exercise feedback system 100 determines 720 a first classification of a first subset of the physiological data. The first classification is selected by a model (e.g., adaptation model 310 of FIG. 3) from a set of multiple classifications each representing a type of a physiological adaptation (e.g., power, strength, hypertrophy, endurance, and speed). The model is trained to determine, for each of the types of physiological adaptations, a probability that a given subset of physiological data is associated with the physiological adaptation.

The model determines 730 using at least the first classification, a second classification from the set of classifications of a second subset of the physiological data. In some embodiments, motion data from the sensors of the garment are also provided as input to the model, e.g., to determine that the first and second subsets of the physiological data correspond to periods of time during which the user actively performed at least a portion of the one or more exercises. In some embodiments, the model determines the classifications responsive to determining that a noise level of the sensors of the garment is less than a threshold noise level. The noise level may be based on bioimpedance, e.g., quality of physical contact between the sensors and skin of the user.

The exercise feedback system 100 transmits 740 biofeedback to a client device 110 for presentation to the user. Additionally or alternatively, biofeedback and/or associated classifications/calculations can be stored and transmitted 740 by the exercise feedback system 100 to a cloud-based computing system, for presentation on another client device (e.g., associated with a coaching entity), as shown in FIG. 7. The biofeedback is generated using at least the first classification and the second classification. In some embodiments, the exercise feedback system 100 modifies a workout based on the biofeedback or classifications. For example, if the athlete's goal is to improve power adaptation and the athlete's metrics are showing that a greater portion of the athlete's training is targeting the strength adaptation, the exercise feedback system 100 can recommend additional sets with decreased weight and at higher velocity to target power adaptation. Additionally, the exercise feedback system 100 can add exercises to a workout plan for the athlete to target a power adaptation such as max vertical jump or max broad jump. In a different use case, the exercise feedback system 100 may reduce volume, intensity, or frequency of workout to mitigate fatigue of an athlete, and reduce injury risk. Additionally or alternatively, the exercise feedback system 100 can modify any number of parameters for a user's training plan, where parameters can be associated with one or more of: type of exercise, number of sets for an exercise, number of repetitions, recommended weight settings, recommended intensity settings, recommended volume settings, recommended frequency settings, prescribed rest settings (e.g., number of rests, duration of rests, etc.), in order to influence type of adaptation for the user (e.g., in relation to alignment with user goals). Additionally or alternatively, the exercise feedback system 100 can determine one or more of: rest and recovery intervals where, for instance, power and strength adaptations take relatively more time to recover from than hypertrophy/endurance adaptations. Additionally or alternatively, in other applications, outputs of the exercise feedback system 100 can be used to match training with user preferences in activities (e.g., sports) and/or life. For instance, for a basketball player, the exercise feedback system 100 can generate a training regimen matched to an adaptation distribution of 70% endurance, 20% power, and 10% strength for the basketball player. In other examples, the exercise feedback system 100 can generate a training regimen matched to other adaptation profiles with distributions of endurance, power, strength, and/or other aspects.

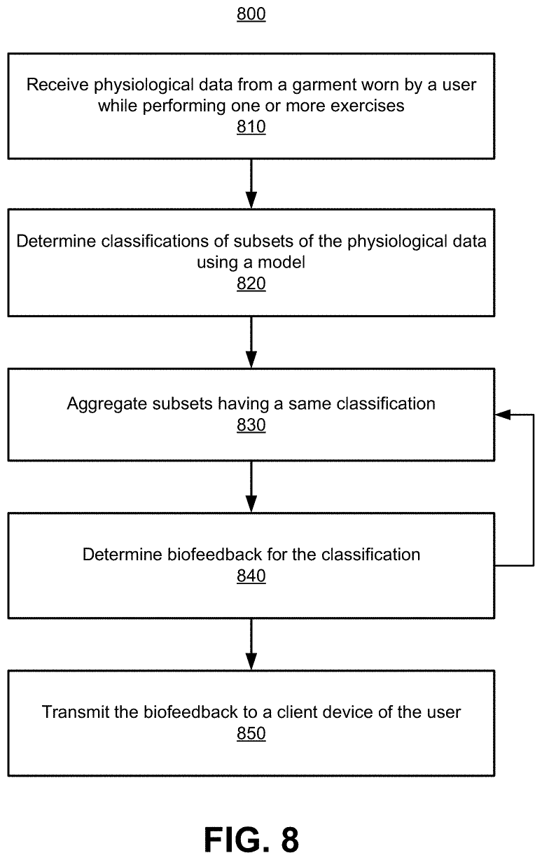

FIG. 8 is another flowchart of a process 800 for providing biofeedback, according to an embodiment. In some embodiments, the process 800 is performed by the exercise feedback system 100—e.g., modules of the exercise feedback system 100 described with reference to FIG. 3—within the system environment in FIG. 1. The process 800 may include different or additional steps than those described in conjunction with FIG. 8 in some embodiments or perform steps in different orders than the order described in conjunction with FIG. 8.

In one embodiment, the exercise feedback system 100 receives 810 physiological data from a garment worn by a user. The physiological data describes muscle activation of a set of muscles of the user while performing one or more exercises. The garment includes a set of sensors configured to generate the physiological data. The exercise feedback system 100 determines 820 classifications of subsets of the physiological data. The classifications are selected by a model (e.g., adaptation model 310 of FIG. 3) from a set of multiple classifications each representing a type of a physiological adaptation. The model trained to determine, for each of the types of physiological adaptations, a probability that a given subset of physiological data is associated with the physiological adaptation.

The exercise feedback system 100 aggregates 830 subsets having a same classification to determine 840 biofeedback for each of the set of classifications. The exercise feedback system 100 may repeat steps 830-840 for any number of classifications, e.g., each of which corresponding to a different physiological adaptation. The exercise feedback system 100 transmits 850 the biofeedback for each of the plurality of classifications to a client device for presentation to the user.

As an example of biofeedback, the adaptation model 310 determines a criteria for detecting or evaluating a risk of injury. The risk of injury may be determined based on an identified imbalance of muscle stress or a fatigue level of an athlete, and the biofeedback engine 330 provides context informing the athlete to address the identified imbalance. For instance, the biofeedback recommends increasing training on the left or right side upper arm muscles or reducing the amount of weights overall to recover from a high-risk fatigue level and avoid injury. In some embodiments, the adaptation model 310 calculates a ratio of contribution between two (or more) specific muscles, and determines that there is a risk injury responsive to determining that the ratio is greater than a threshold, which is indicative of a more severe imbalance or discrepancy in exercise form relative to the correct form.

In some variations, the method can further include one or more of: implementing a history of activity sessions of the user to refine characterization of at least one type of the physiological adaptation; implementing a contextual input from the user to refine characterization of at least one type of the physiological adaptation, the contextual input describing difficulty in performing an activity; and customizing a characterization of at least one type of the physiological adaptation to a demographic comprising the user.

### VI. Additional Considerations

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments of the invention may also relate to a product that is produced by a computing process described herein. Such a product may include information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A method comprising:
receiving physiological data from a garment worn by a user, the physiological data describing muscle activation of a plurality of muscles of the user while performing one or more exercises, the garment including a plurality of sensors configured to generate the physiological data;
determining a first classification of a first subset of the physiological data, the first classification selected by a model from a plurality of classifications each representing a type of a physiological adaptation, the model trained to determine, for each of the types of physiological adaptations, a probability that a given subset of physiological data is associated with the physiological adaptation;
determining, by the model using at least the first classification, a second classification from the plurality of classifications of a second subset of the physiological data; and
transmitting biofeedback to at least one of a client device for presentation to the user and a coaching device, the biofeedback generated using at least the first classification and the second classification.

2. The method of claim 1, further comprising:
receiving motion data and electrocardiogram data from at least one of the plurality of sensors; and
providing the motion data and electrocardiogram data as inputs to the model for determining the first and second classifications.

3. The method of claim 2, further comprising receiving respiration data from at least one of the plurality of sensors; and providing the respiration data as an input to the model for determining the first and second classifications.

4. The method of claim 2, further comprising:
determining, using the motion data, that the first and second subsets of the physiological data correspond to periods of time during which the user actively performed at least a portion of the one or more exercises.

5. The method of claim 1, further comprising:
determining at least one metric for each of the plurality of muscles using the first and second classifications; and
generating the biofeedback by aggregating the metrics of the plurality of muscles.

6. The method of claim 5, wherein determining the at least one metric comprises:
determining a plurality of subsets of the physiological data classified with a same one of the types of physiological adaptations;
determining a training load experienced by a muscle of the plurality of muscles for each of the plurality of subsets of the physiological data; and
determining, as the at least one metric, an aggregate training load for the muscle by aggregating each of the training loads.

7. The method of claim 6, further comprising:
determining an injury risk of the user responsive to determining that the aggregate training load is greater than a threshold load for a certain period of time.

8. The method of claim 1, further comprising:
determining a noise level based on bioimpedance between the plurality of sensors and skin of the user; and
wherein determining the first classification is responsive to determining that the noise level is less than a threshold noise level.

9. The method of claim 1, further comprising:
receiving electromyography data from at least one of the plurality of sensors;
determining a rate of change of the electromyography data; and
determining, by the model, one or more of a power, a strength, and a hypertrophy physiological adaptation as the first classification responsive to determining that the rate of change of the electromyography data is greater than a threshold rate.

10. The method of claim 9, further comprising:
selecting, by the model, the one or more of the power, the strength, and the hypertrophy physiological adaptation as the first classification according to an amplitude of the electromyography data.

11. The method of claim 1, further comprising:
determining, by the model, one or more of a speed and an endurance physiological adaptation as the first classification responsive to identifying a repeated pattern in at least one of motion data and electromyography data received from at least one of the plurality of sensors.

12. A method comprising:
creating a training set upon applying a set of operations to a reference set of physiological data generated from one or more reference users, the set of operations comprising at least one of a frequency-domain analysis and a filtering operation;
training a model, using the training set, to determine, for each of a set of types of physiological adaptations, a probability that a given subset of physiological data is associated with one or more of the set of types of physiological adaptations;
collecting user physiological data from a garment worn by a user, the user physiological data describing muscle activation of a plurality of muscles of the user while performing one or more exercises, the garment including a plurality of sensors configured to generate the physiological data;
determining a classification of a subset of the user physiological data upon applying the model to the user physiological data;
determining biofeedback corresponding to the classification by aggregating subsets of the user physiological data having the classification; and
transmitting the biofeedback to a client device for presentation to the user.

13. The method of claim 12, further comprising:
receiving motion data from at least one of the plurality of sensors;
determining a noise level based on bioimpedance between the plurality of sensors and skin of the user; and
wherein determining the classification is responsive to:
determining, using the motion data, that the subset of the user physiological data corresponds to a period of time during which the user actively performed at least a portion of the one or more exercises; and
determining that the noise level is less than a threshold noise level.

14. A computer program product comprising a non-transitory computer readable storage medium having instructions encoded thereon that, when executed by a processor, cause the processor to:
receive physiological data from a garment worn by a user, the physiological data describing muscle activation of a plurality of muscles of the user while performing one or more exercises, the garment including a plurality of sensors configured to generate the physiological data;
determine a first classification of a first subset of the physiological data, the first classification selected by a model from a plurality of classifications each representing a type of a physiological adaptation, the model trained to determine, for each of the types of physiological adaptations, a probability that a given subset of physiological data is associated with the physiological adaptation;
determine, by the model using at least the first classification, a second classification from the plurality of classifications of a second subset of the physiological data; and
transmit biofeedback to a client device for presentation to the user, the biofeedback generated using at least the first classification and the second classification.

15. The non-transitory computer readable storage medium of claim 14, having further instructions that when executed by the processor cause the processor to:
receive motion data from at least one of the plurality of sensors; and
provide the motion data as input to the model for determining the first and second classifications.

16. The non-transitory computer readable storage medium of claim 15, having further instructions that when executed by the processor cause the processor to:
determine, using the motion data, that the first and second subsets of the physiological data correspond to periods of time during which the user actively performed at least a portion of the one or more exercises.

17. The non-transitory computer readable storage medium of claim 14, having further instructions that when executed by the processor cause the processor to:
determine at least one metric for each of the plurality of muscles using the first and second classifications; and
generate the biofeedback by aggregating the metrics of the plurality of muscles.

18. The non-transitory computer readable storage medium of claim 17, wherein determining the at least one metric comprises:
determine a plurality of subsets of the physiological data classified with a same one of the types of physiological adaptations;
determine a training load experienced by a muscle of the plurality of muscles for each of the plurality of subsets of the physiological data; and
determine, as the at least one metric, an aggregate training load for the muscle by aggregating each of the training loads.

19. The non-transitory computer readable storage medium of claim 14, having further instructions that when executed by the processor cause the processor to:
determine a noise level based on bioimpedance between the plurality of sensors and skin of the user; and
wherein determining the first classification is responsive to determining that the noise level is less than a threshold noise level.

20. The non-transitory computer readable storage medium of claim 14, having further instructions that when executed by the processor cause the processor to:
receive electromyography data from at least one of the plurality of sensors;
determine a rate of change of the electromyography data; and
determine, by the model, one or more of a power, a strength, and a hypertrophy physiological adaptation as the first classification responsive to determining that the rate of change of the electromyography data is greater than a threshold rate.

21. The non-transitory computer readable storage medium of claim 14, having further instructions that when executed by the processor cause the processor to:
determine, by the model, one of a speed and an endurance physiological adaptation as the first classification responsive to identifying a repeated pattern in at least one of motion data and electromyography data received from at least one of the plurality of sensors.

22. The non-transitory computer readable storage medium of claim 14, having further instructions that when executed by the processor cause the processor to perform at least one of:
implementing a history of activity sessions of the user to refine characterization of at least one type of the physiological adaptation;
implementing a contextual input from the user to refine characterization of at least one type of the physiological adaptation, the contextual input describing difficulty in performing an activity; and
customizing a characterization of at least one type of the physiological adaptation to a demographic comprising the user.
